(19) United States
(12) Patent Application Publication
(10) Pub. No.: US 2017/0296491 A1
(43) Pub. Date: Oct. 19, 2017

(54) PHARMACEUTICAL COMPOSITIONS COMPRISING LEVODOPA AMIDE AND USES THEREOF

(71) Applicant: NeuroDerm, Ltd., Rehovot (IL)

(72) Inventors: Oron Yacoby-Zeevi, Moshav Bitsaron (IL); Mara Nemas, Gedera (IL); Eduardo Zawoznik, Mazkeret Batya (IL); Irena Vainshtok, Rehovot (IL); Einat Sela, Rehovot (IL)

(21) Appl. No.: 15/360,165

(22) Filed: Nov. 23, 2016

Related U.S. Application Data
(60) Provisional application No. 62/259,324, filed on Nov. 24, 2015.

(51) Int. Cl. A61K 31/165; A61K 31/198; A61K 45/06; A61K 47/02

(52) U.S. Cl. CPC A61K 31/165; A61K 47/02; A61K 31/198; A61K 45/06

(57) ABSTRACT

The present invention discloses various aqueous pharmaceutical compositions comprising a levodopa amide compound, or a salt thereof, which are stable for at least 24 hours at room temperature, and use thereof in treatment of diseases or disorders characterized by neurodegeneration and/or reduced levels of brain dopamine, e.g., Parkinson's disease.

### CROSS-REFERENCE TO RELATED APPLICATIONS

This application claims priority to and the benefit of U.S. Provisional Patent Application No. 62/259,324, filed Nov. 24, 2015, the entire content of which being herewith incorporated by reference as if fully disclosed herein.

### TECHNICAL FIELD

The present invention relates to pharmaceutical compositions comprising a levodopa amide (LDA) compound, or a salt thereof, and use thereof for treating diseases and disorders characterized by neurodegeneration and/or reduced levels of brain dopamine, e.g., Parkinson's disease.

### BACKGROUND ART

Parkinson's disease is a degenerative condition characterized by reduced concentration of the neurotransmitter dopamine in the brain. Levodopa (L-dopa or L-3,4-dihydroxyphenylalanine) is an immediate metabolic precursor of dopamine that, unlike dopamine, is able to cross the blood brain barrier, and is most commonly used for restoring the dopamine concentration in the brain. For the past 40 years, levodopa has remained the most effective therapy for the treatment of Parkinson's disease.

However, conventional treatments for Parkinson's disease with L-DOPA have proven to be inadequate for many reasons of record in the medical literature. For example, some patients become less responsive to levodopa such that previously effective doses eventually fail to produce any therapeutic benefit; and the systemic administration of levodopa, although producing clinically beneficial effects at first, is complicated by the need to increase the dosages that may result in adverse side effects. For such reasons, the benefits of levodopa treatment often begin to diminish after about 3 or 4 years of therapy, irrespective of the initial therapeutic response.

The peripheral administration of levodopa is further complicated by the fact that only about 1-3% of the levodopa administered actually enters the brain unaltered, the remainder being metabolized extracerebrally, predominantly by decarboxylation to dopamine. Dopamine does not penetrate the blood brain barrier. The metabolic transformation of levodopa to dopamine is catalyzed by the aromatic L-amino acid decarboxylase enzyme, a ubiquitous enzyme with particularly high concentrations in the intestinal mucosa, liver, brain and brain capillaries. Due to the possibility of extracerebral metabolism of levodopa, it is necessary to administer large doses of levodopa leading to high extracerebral concentrations of dopamine. The co-administration of LD and a peripheral DOPA decarboxylase (aromatic L-amino acid decarboxylase) inhibitor such as carbidopa or benserazide has been found to reduce the dosage requirements of LD and, respectively, some of the side effects, although not sufficiently.

Finally, certain fluctuations in clinical response to levodopa occur with increasing frequency as treatment continues. In some patients, these fluctuations relate to the timing of levodopa intake, and they are then referred to as wearing-off reactions or end-of-dose akinesia. In other instances, fluctuations in clinical state are unrelated to the timing of doses (on-off phenomenon). In the on-off phenomenon, off-periods of marked akinesia and bradykinesia alternate over the course of a few hours with on-periods of improved mobility which are often associated with troublesome dyskinesia.

It is well accepted in the art that many of the problems recited above result from the unfavorable pharmacokinetic properties of L-DOPA and, more particularly, from its poor water solubility, bioavailability and fast degradation in vivo. Thus, there is still an urgent need for effective therapeutic formulations for treating disorders such as Parkinson's disease.

U.S. Pat. No. 8,048,926 discloses L-DOPA amide derivatives (referred to as L-DOPA prodrugs), pharmaceutical compositions comprising them, and their use in the treatment of conditions associated with impaired dopaminergic activity/signaling, e.g., Parkinson's disease. The compounds disclosed are said to be characterized by high permeability through the blood brain barrier.

### SUMMARY OF INVENTION

In one aspect, the present invention provides a an aqueous pharmaceutical composition, also referred to herein as “pharmaceutical composition A”, having a pH of about 3 to about 7 at 25° C., said composition comprising a levodopa amide (LDA) compound of the general formula I:

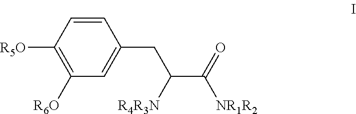

or an enantiomer, diastereomer, or racemate thereof,
wherein
R₁, R₂, R₃ and R₄ each independently is H, (C₁-C₆)alkyl, (C₂-C₆)alkenyl, (C₂-C₆)alkynyl, cycloalkyl, aryl, —O—C(═O)—R′, —C(═O)—OR′, —C(═O)—R′, —C(═S)—R′, —O—C(═O)—NR′R′, —O—C(═S)—NR′R′, or —O—C(═O)—R″, or R₁ and R₂ together with the nitrogen atom to which they are attached form a 5- or 6-membered ring, or R₃ and R₄, together with the nitrogen atom to which they are attached form a 5- or 6-membered ring; and
R₅ and R₆ each independently is H, (C₁-C₃)alkyl, cycloalkyl, phenyl, or —P(═O)(OR′)₂,
R′ each independently is H, (C₁-C₆)alkyl, (C₂-C₆)alkenyl, cycloalkyl, aryl, or heteroaryl bonded through a ring carbon; and
R″ is a saturated or unsaturated hydrocarbon chain having at least 10 carbon atoms, and
an organic- or inorganic-acid having n acidic groups, wherein n is an integer of 1 or more,
wherein the molar ratio of said LDA compound to said acid is about 1:1/n to about 1:≧1.1, and said composition is stable for at least 24 hours at room temperature.

The pharmaceutical composition A defined above may further comprise a decarboxylase inhibitor such as carbidopa, and at least one of a basic amino acid, e.g., arginine, or an amino sugar, e.g., meglumine; and optionally at least one of a buffer, an antioxidant, an additional active agent such as a catechol-O-methyl transferase (COMT) inhibitor or a monoamine oxidase (MAO) inhibitor, and a surfactant.

In another aspect, the present invention provides an aqueous pharmaceutical composition, also referred to herein as “pharmaceutical composition B”, having a pH of about 3 to about 9.5, or about 4 to about 8, or about 5 to about 7, or about 5.5 to about 6.5, at 25° C., said composition comprising a salt of a LDA compound of the general formula I as defined above, or an enantiomer, diastereomer, or racemate thereof, a decarboxylase inhibitor, e.g., carbidopa, or a salt thereof, and optionally at least one of a basic amino acid, e.g., arginine, or an amino sugar, e.g., meglumine,
wherein the weight ratio of said decarboxylase inhibitor to said salt of LDA compound is about 1:1 to about 1:100, about 1:2 to about 1:60, about 1:4 to about 1:40, or about 1:10 to about 1:40; and the molar ratio or said decarboxylase inhibitor or salt thereof to said basic amino acid or said amino sugar is about 1:1 to about 1:4, or about 1:1 to about 1:3.5, or about 1:1 to about 1:2.5, and
wherein said composition is stable for at least 24 hours at room temperature.

The pharmaceutical composition B defined above may further comprise at least one of a buffer, an antioxidant, an additional active agent such as a COMT inhibitor or a MAO inhibitor, and a surfactant.

In yet another aspect, the present invention provides an aqueous pharmaceutical composition, also referred to herein as “pharmaceutical composition C”, having a pH of about 3 to about 6, or about 4 to about 5.5, at 25° C., said composition comprising a salt of a LDA compound of the general formula I as defined above, or an enantiomer, diastereomer, or racemate thereof, and a buffer,
wherein said composition is stable for at least 24 hours at room temperature.

The pharmaceutical composition C defined above may further comprise at least one of an antioxidant, an additional active agent such as a COMT inhibitor or a MAO inhibitor, and a surfactant.

In particular embodiments, the LDA compound comprised, either per se or as a salt thereof, within each one of the aqueous pharmaceutical compositions of the present invention is 2-amino-3-(3,4-dihydroxyphenyl)propanamide, or an enantiomer, diastereomer, or racemate thereof.

The pharmaceutical compositions disclosed herein are useful for treatment of diseases or disorders characterized by neurodegeneration and/or reduced levels of brain dopamine. Such diseases and disorders include neurological or movement diseases or disorders selected from restless leg syndrome, Parkinson's disease, secondary parkinsonism, Huntington's disease, Parkinson's like syndrome, progressive supranuclear palsy (PSP), multiple system atrophy (MSA), amyotrophic lateral sclerosis (ALS), Shy-Drager syndrome, dystonia, Alzheimer's disease, Lewy body disease (LBD), akinesia, bradykinesia, and hypokinesia; conditions resulting from brain injury including carbon monoxide or manganese intoxication; and conditions associated with a neurological disease or disorder including alcoholism, opiate addiction, and erectile dysfunction. In a particular embodiment, the disease treated with the pharmaceutical compositions of the invention is Parkinson's disease.

In a further aspect, the present invention thus relates to a method for treatment of a disease or disorder characterized by neurodegeneration and/or reduced levels of brain dopamine, comprising administering to a patient, e.g., an individual, in need thereof a therapeutically effective amount of a pharmaceutical composition A as defined above, provided that said composition comprises a decarboxylase inhibitor and at least one of a basic amino acid or an amino sugar; or a pharmaceutical composition B as defined above.

In yet a further aspect, the present invention relates to a method for treatment of a disease or disorder characterized by neurodegeneration and/or reduced levels of brain dopamine, comprising co-administering to a patient, e.g., an individual, in need thereof (i) a first pharmaceutical composition selected from a pharmaceutical composition A as defined above, provided that said composition does not comprise a decarboxylase inhibitor or a salt thereof, or a pharmaceutical composition C as defined above; and (ii) a second pharmaceutical composition comprising a decarboxylase inhibitor and optionally at least one of a basic amino acid or an amino sugar; and/or a COMT inhibitor; and/or a MAO inhibitor.

In still a further aspect, the present invention provides a kit comprising (i) a first pharmaceutical composition selected from a pharmaceutical composition A as defined above, provided that said composition comprises neither a decarboxylase inhibitor nor a salt thereof, or a pharmaceutical composition C as defined above; (ii) a second pharmaceutical composition comprising a decarboxylase inhibitor or a salt thereof, and optionally at least one of a basic amino acid or an amino sugar, and/or a COMT inhibitor; and/or a MAO inhibitor; and (iii) optionally instructions for co-administration of said pharmaceutical compositions for treatment of a disease or disorder characterized by neurodegeneration and/or reduced levels of brain dopamine.

### BRIEF DESCRIPTION OF THE FIGURES

FIG. 1 depicts the plasma concentrations of LDA and LD in CD-1 mice plasma after Intravenous administration of LDA-HCl (20 mg/kg).

FIG. 2 depicts the plasma concentrations of LDA and LD in CD-1 mice plasma after Oral administration of LDA-HCl (20 mg/kg).

FIG. 3 depicts the plasma concentrations of LDA and LD in CD-1 mice after continuous subcutaneous administration of 170 mg/m1 of LDA-HCl at a rate of 0.5 μl/hr for 3 days.

FIG. 4 depicts LD plasma concentration following oral administration of 25 mg/kg LDA-HCl with or without 10 mg/kg CD in rats.

FIG. 5 depicts LDA plasma concentration following oral administration of 25 mg/kg LDA-HCl with or without 10 mg/kg CD in rats.

FIG. 6 depicts LD plasma concentrations following oral administration of 25 mg/kg LD with 10 mg/kg CD in rats.

### DETAILED DESCRIPTION OF THE INVENTION

Levodopa amides of the general formula I are neutral compounds at physiological pH. In other words, as opposed to L-DOPA that has a zwitterionic form, the carboxylic group in a corresponding amide is neutralized, rendering such compounds less hydrophilic and thereby more membrane permeable. Amides are hydrolyzed in vivo by amido peptidase, wherein the rate of the enzymatic hydrolysis is determined by the nature of the hydrolyzed bond. Amides are known as much more stable molecules than esters and salts, and the hydrolysis rate of amides by amido peptidases is therefore significantly reduced as compared with the corresponding hydrolysis of ester or salts. Considering the above, it was suggested at the time (Atlas, Dopamide: novel, water-soluble, slow-release L-dihydroxyphenylalanine (L-DOPA) precursor moderates L-DOPA conversion to dopamine and generates a sustained level of dopamine at dopaminergic neurons. CNS Neuroscience & Therapeutics, 2016, 22, 461-467) that the rate of hydrolysis of an amide derivative of L-DOPA in the periphery would be substantially reduced, providing for enhanced accumulation thereof in the brain.

The same feature was also suggested to apply for brain derived, endogenous amido peptidases, such that once the L-DOPA amide derivative penetrates the blood brain barrier, the rate of its conversion into L-DOPA is relatively slow. It was further suggested that this feature may result in the gradual formation of L-DOPA, mimicking a slow release effect of the drug, and that the slow hydrolysis of amides together with its enhanced blood brain barrier permeability may enable the administration of lower doses of L-DOPA amide derivatives to produce clinically meaningful effects with reduced adverse side effects and prolonged treatment period.

As surprisingly found in accordance with the present invention, LDA does not have a “slow release” effect, but rather it is rapidly metabolized to levodopa, thus requiring the co-administration/co-formulation of LDA and a decarboxylase inhibitor and/or a COMT inhibitor to improve the pharmacokinetic of levodopa.

Provided herein, in general, pharmaceutical compositions comprising a levodopa amide compound or a derivative thereof, or a pharmaceutically acceptable salt thereof, and a pharmaceutically acceptable excipient, e.g., for use in treating patients with suffering from a disease or disorder characterized by neurodegeneration and/or reduced levels of brain dopamine, more particularly a neurological or movement disorder such as Parkinson's disease.

More particularly, in one aspect, the present invention provides a an aqueous pharmaceutical composition, also referred to herein as “pharmaceutical composition A”, having a pH of about 3 to about 7, e.g., about 3 to about 4, about 4 to about 5, about 5 to about 6, or about 6 to about 7, at 25° C., said composition comprising a LDA compound of the general formula I:

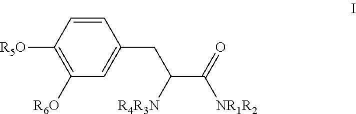

or an enantiomer, diastereomer, or racemate thereof,
wherein
R₁, R₂, R₃ and R₄ each independently is H, (C₁-C₆)alkyl, (C₂-C₆)alkenyl, (C₂-C₆)alkynyl, cycloalkyl, aryl, —O—C(═O)—R′, —C(═O)—OR′, —C(═O)—R′, —C(═S)—R′, —O—C(═O)—NR′R′, —O—C(═S)—NR′R′, or —O—C(═O)—R″, or R₁ and R₂ together with the nitrogen atom to which they are attached form a 5- or 6-membered ring, or R₃ and R₄, together with the nitrogen atom to which they are attached form a 5- or 6-membered ring; and
R₅ and R₆ each independently is H, (C₁-C₃)alkyl, cycloalkyl, phenyl, or —P(═O)(OR′)₂,
R′ each independently is H, (C₁-C₆)alkyl, (C₂-C₆)alkenyl, cycloalkyl, aryl, or heteroaryl bonded through a ring carbon; and
R″ is a saturated or unsaturated hydrocarbon chain having at least 10 carbon atoms, and
an organic- or inorganic-acid having n acidic groups such as carboxylic, phosphonic, phosphinic, sulphonic, or sulphinic groups, wherein n is an integer of 1 or more,
wherein the molar ratio of said LDA compound to said acid is about 1:1/n to about 1:≧1.1, and said composition is stable for at least 24 hours, e.g., for at least 24, 48, 72 or 96 hours, at least 1, 2 or 3 weeks, at least 1, 2 or 3 months, or at least 1 year, at room temperature or at −20 to −80° C.

The term “alkyl” as used herein means a straight or branched saturated hydrocarbon radical having 1-6 carbon atoms and includes, e.g., methyl, ethyl, n-propyl, isopropyl, n-butyl, sec-butyl, isobutyl, tert-butyl, n-pentyl, isoamyl, 2,2-dimethylpropyl, n-hexyl, and the like. Preferred are (C₁-C₃)alkyl groups, more preferably methyl and ethyl. The alkyl group may be unsubstituted or substituted.

The terms “alkenyl” and “alkynyl” as used herein mean straight and branched hydrocarbon radicals having 2-6 carbon atoms and one or more double or triple bonds, respectively, and include ethenyl, propenyl, 3-buten-1-yl, 2-ethenylbutyl, and the like, and propynyl, 2-butyn-1-yl, 3-pentyn-1-yl, 3-hexynyl, and the like. C₂-C₃ alkenyl and alkynyl radicals are preferred, more preferably C₂ alkenyl and alkynyl.

The term “cycloalkyl” as used herein means a cyclic or bicyclic hydrocarbyl group having 3-10 carbon atoms such as cyclopropyl, cyclobutyl, cyclopentyl, cyclohexyl, cycloheptyl, cyclooctyl, adamantyl, bicyclo[3.2.1]octyl, bicyclo[2.2.1]heptyl, and the like. Preferred are (C₅-C₁₀)cycloalkyls, more preferably (C₅-C₇)cycloalkyls. The cycloalkyl group may be unsubstituted or substituted.

The term “aryl” as used herein denotes an aromatic carbocyclic group having 6-14 carbon atoms consisting of a single ring or multiple rings either condensed or linked by a covalent bond such as, but not limited to, phenyl, naphthyl, phenanthryl, and biphenyl. The aryl group may be unsubstituted or substituted.

The term “heteroaryl” as used herein refers to a monocyclic or fused ring (i.e., rings which share an adjacent pair of atoms) group having one or more atoms selected from nitrogen, oxygen and sulfur, and a completely conjugated pi-electron system. Non-limiting examples of heteroaryl groups include pyrrole, furane, thiophene, imidazole, oxazole, thiazole, pyrazole, pyridine, pyrimidine, quinoline, isoquinoline and purine.

The term “halo” refers to a fluorine, chlorine, bromine or iodine atom.

In certain embodiments, the LDA compound comprised, either per se or as a salt thereof, within the pharmaceutical composition of the present invention (pharmaceutical composition A, B or C) is a compound of the general formula I, or an enantiomer, diastereomer, or racemate thereof, wherein R₁, R₂, R₃ and R₄ each is H; or one of R₁ and R₂, and/or one of R₃ and R₄, each is —C(═O)—R′ wherein R′ is (C₁-C₆)alkyl, preferably (C₁-C₃)alkyl such as methyl or ethyl, and the others of R₁, R₂, R₃ and R₄ each is H. In particular such embodiments, R₁, R₂, R₃ and R₄ each is H; or one of R₃ and R₄ is —C(═O)—(C₁-C₆)alkyl, preferably —C(═O)—(C₁-C₃)alkyl such as —C(=O)-methyl or —C(═O)-ethyl, and the others of R₁, R₂, R₃ and R₄ each is H.

In certain embodiments, the LDA compound comprised, either per se or as a salt thereof, within the pharmaceutical composition of the present invention (pharmaceutical composition A, B or C) is a compound of the general formula I, or an enantiomer, diastereomer, or racemate thereof, wherein R₅ and R₆ each independently is (C₁-C₃)alkyl, preferably methyl or ethyl, or H. In particular such embodiments, R₅ and R₆ each is H.

In certain embodiments, the LDA compound comprised, either per se or as a salt thereof, within the pharmaceutical composition of the present invention (pharmaceutical composition A, B or C) is a compound of the general formula I, wherein (i) R₁, R₂, R₃ and R₄ each is H; or one of R₁ and R₂, and/or one of R₃ and R₄, each is —C(═O)—(C₁-C₆)alkyl, preferably —C(═O)—(C₁-C₃)alkyl, and the others of R₁, R₂, R₃ and R₄ each is H; and (ii) R₅ and R₆ each independently is (C₁-C₃)alkyl, preferably methyl or ethyl, or H. In particular such embodiments, (i) R₁, R₂, R₃, R₄, R₅ and R₆ each is H; or one of R₃ and R₄ is —C(═O)—(C₁-C₆)alkyl, preferably —C(═O)—(C₁-C₃)alkyl, and the others of R₁, R₂, R₃ and R₄, as well as R₅ and R₆, each is H.

In certain particular embodiments, the LDA compound comprised, either per se or as a salt thereof, within the pharmaceutical composition of the invention is 2-amino-3-(3,4-dihydroxyphenyl)propanamide of formula II (Table 1), i.e., a LDA compound of the formula I, wherein R₁, R₂, R₃, R₄, R₅ and R₆ are each H, or an enantiomer, diastereomer, or racemate thereof. In other particular embodiments, the LDA compound comprised, either per se or as a salt thereof, within the pharmaceutical composition of the invention is 2-acetamido-3-(3,4-dihydroxyphenyl)propanamide of formula III (Table 1), i.e., a LDA compound of the formula I, wherein R₃ is acetyl; and R₁, R₂, R₄, R₅, R₆ are each H, or an enantiomer, diastereomer, or racemate thereof.

TABLE 1
Specific compounds of the general formula I described herein

| Name | Structure |
|---|---|
| 2-amino-3- (3,4-dihydroxyphenyl) propanamide (formula II) | 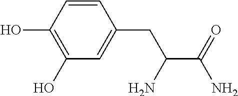 [SMILES: NC(=O)C(N)Cc1ccc(O)c(O)c1] |
| 2-acetamido-3- (3,4-dihydroxyphenyl) propanamide (formula III) | 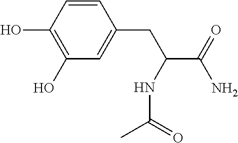 [SMILES: CC(=O)NC(Cc1ccc(O)c(O)c1)C(N)=O] |

In certain embodiments, the pharmaceutical composition of the present invention comprises about 1% or more, e.g., about 1% or 5%, to about 20%, 25%, 30%, or more, by weight of said LDA compound or salt thereof.

The acid comprised within the pharmaceutical composition A of the present invention can be an organic acid, an inorganic acid, or any combination thereof. Examples of suitable organic acids include, without being limited to, methanesulfonic acid, ethanesulfonic acid, benzenesulfonic acid, p-toluenesulfonic acid, maleic acid, malic acid, fumaric acid, tartaric acid, benzoic acid, acetic acid, citric acid, ascorbic acid, lactic acid, gluconic acid, formic acid, oxalic acid, succinic acid, or acidic amino acids such as glutamic acid and aspartic acid. Examples of suitable inorganic acids include, without limiting, hydrochloric acid, hydrobromic acid, phosphoric acid, sulfuric acid, and carbonic acid. In particular embodiments exemplified herein, the acid comprised within the pharmaceutical composition of the invention is hydrochloric acid, succinic acid, glutamic acid, citric acid, tartaric acid or acetic acid.

According to the present invention, the molar ratio of said LDA compound to said acid in the pharmaceutical composition A of the present invention is about 1:1/n to about 1:≧1.1, wherein n is an integer of 1 or more representing the number of acidic groups in said acid. The molar ratio of said LDA compound to said acid may thus be in a range of about 1:1 to about 1:≧1.1 (e.g., about 1:1.1, 1:1.2, 1:1.3, 1:1.4, 1:1.5, 1:1.6, 1:1.7, 1:1.8, 1:1.9, 1:2, or 1:≧2) when n is 1 (in the case of, e.g., methanesulfonic acid, ethanesulfonic acid, benzenesulfonic acid, p-toluenesulfonic acid, benzoic acid, acetic acid, lactic acid, gluconic acid, formic acid, hydrochloric acid, hydrobromic acid, phosphoric acid, and carbonic acid); about 1:0.5 to about 1:≧1.1 (e.g., about 1:1.1, 1:1.2, 1:1.3, 1:1.4, 1:1.5, 1:1.6, 1:1.7, 1:1.8, 1:1.9, 1:2, or 1:≧2) when n is 2 (in the case of, e.g., maleic acid, malic acid, fumaric acid, tartaric acid, oxalic acid, succinic acid, glutamic acid, aspartic acid, and sulfuric acid); or about 1:0.33 to about 1:≧1.1 (e.g., about 1:1.1, 1:1.2, 1:1.3, 1:1.4, 1:1.5, 1:1.6, 1:1.7, 1:1.8, 1:1.9, 1:2, or 1:≧2) when n is 3 (in the case of, e.g., citric acid). For example, provided herein is a composition wherein the molar ratio of LDA to acid is about 1:0.01 to about 1:1.2, or about 1:0.5 to about 1.1, or about 1:0.01 to about 1:1.1.

In certain embodiments, the pharmaceutical composition A of the present invention, as defined in any one of the embodiments above, further comprises a decarboxylase inhibitor, aimed at inhibiting an undesired enzymatic decarboxylation of levodopa to dopamine in the periphery, and either one of, or at least one of, a basic amino acid or an amino sugar. The decarboxylase inhibitor may be selected from carbidopa, benserazide, or a salt thereof, e.g., the arginine-, histidine-, or lysine-salt of carbidopa; the basic amino acid may be selected from arginine, histidine, or lysine; and the amino sugar may be selected from meglumine, D-glucosamine, sialic acid, N-acetylglucosamine, galactosamine, or a combination thereof. Particular such pharmaceutical compositions comprise carbidopa, and either arginine or meglumine. More particular such compositions are those wherein the LDA compound is the compound of formula II or III, or an enantiomer, diastereomer, or racemate thereof, e.g., such compositions which comprise about 1% or 5%, to about 20%, 25%, or 30%, by weight of said LDA compound. In particular such embodiments, the weight ratio of said decarboxylase inhibitor to said LDA compound is about 1:1 to about 1:100, about 1:2 to about 1:60, about 1:5 to about 1:40, or about 1:10 to about 1:40; or the molar ratio of said decarboxylase inhibitor to said basic amino acid or said amino sugar is about 1:1 to about 1:4, about 1:1 to about 1:3.5, or about 1:1 to about 1:2.5.

In certain embodiments, the pharmaceutical composition A of the present invention, as defined in any one of the embodiments above, further comprises a buffer. Examples of buffers that may be used according to the present invention include, without being limited to, citrate buffer, citric acid buffer, acetate buffer, sodium acetate buffer, acetic acid buffer, tartrate buffer, tartaric acid buffer, phosphate buffer, succinic acid buffer, Tris buffer, glycine buffer, hydrochloric acid buffer, potassium hydrogen phthalate buffer, sodium buffer, sodium citrate tartrate buffer, sodium hydroxide buffer, sodium dihydrogen phosphate buffer, disodium hydrogen phosphate buffer, or a mixture thereof.

In certain embodiments, the pharmaceutical composition A of the present invention, as defined in any one of the embodiments above, further comprises one or more antioxidants. Examples of antioxidants that may be used according to the present invention include, without limiting, ascorbic acid or a salt thereof, e.g., sodium ascorbate, calcium ascorbate, or potassium ascorbate, a cysteine such as L-cysteine and N-acetyl cysteine (NAC), a bisulfite or a salt thereof such as sodium metabisulfite, and glutathione.

In some embodiments, the contemplated antioxidants are tyrosinase inhibitors such as captopril, and/or o-quinone scavengers such as NAC, gluthatione, ascorbic acid or a salt thereof, and/or L-cysteine, and/or Cu⁺² chelators such as Na₂-EDTA and Na₂-EDTA-Ca. In some embodiments, carbidopa may act as an agent that inhibits the formation of oxidation products. In an embodiment, the disclosed compositions may include an agent chosen from methimazole, quercetin, arbutin, aloesin, N-acetylglucoseamine, retinoic acid, alpha-tocopheryl ferulate, Mg ascorbyl phosphate (MAP), substrate analogues (e.g., sodium benzoate, L-phenylalanine), DMSA (succimer), DPA (D-penicillamine), trientine-HCl, dimercaprol, clioquinol, sodium thiosulfate, triethylenetetramine (TETA), tetraethylenepentamine (TEPA), curcumin, neocuproine, tannin, and/or cuprizone. Other contemplated antioxidants that may form part of the disclosed composition include sulfite salts (e.g., sodium hydrogen sulfite or sodium metabisulfite), lipoic acid, CB4 (N-acetyl CysGlyProCys amide), CB3 (N-acetyl CysProCys amide), AD4 (N-acetyl cysteine amide), AD6 (N-acetylGluCysGly amide), AD7 (N-acetylCysGly amide), vitamin E, di-tert-butyl methyl phenols, tert-butyl-methoxyphenols, polyphenols, tocopherols and/or ubiquinones, including but not limited to caffeic acid.

In some embodiments, a disclosed composition comprises about 0.01% to about 1% by weight antioxidant, e.g., about 0.01%, about 0.02%, about 0.03%, about 0.04%, about 0.05%, about 0.06%, about 0.07%, about 0.08%, about 0.09%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, or about 1.0%, by weight antioxidant.

In certain embodiments, the pharmaceutical composition A of the present invention, as defined in any one of the embodiments above, further comprises a COMT inhibitor, or a MAO (either MAO-A or MAO-B) inhibitor. Particular COMT inhibitors include, without limiting, entacapone, tolcapone, and opicapone; and particular MAO inhibitors can be selected from, e.g., moclobemide, rasagiline, selegiline, or safinamide.

In certain embodiments, the pharmaceutical composition A of the present invention, as defined in any one of the embodiments above, further comprises a surfactant. Suitable surfactants include, without being limited to, Tween-80, Tween-60, Tween-40, Tween-20, Tween-65, Tween-85, Span 20, Span 40, Span 60, Span 80, Span 85, polyoxyl 35 castor oil (Cremophor EL), polyoxyethylene-660-hydroxystearate (macrogol 660), or Poloxamer 188 (Pluronic® F-68). Additional one or more pharmaceutically acceptable excipients may be selected from, e.g., N-methylpyrrolidone (NMP), polyvinylpyrrolidone (PVP), and propylene glycol, and added to the composition.

In another aspect, the present invention provides an aqueous pharmaceutical composition, also referred to herein as “pharmaceutical composition B”, having a pH of about 3 to about 9.5, or about 4 to about 8, or about 5 to about 7, or about 5.5 to about 6.5, at 25° C., said composition comprising a salt of a LDA compound of the general formula I as defined above, or an enantiomer, diastereomer, or racemate thereof, a decarboxylase inhibitor or a salt thereof, and at least one of a basic amino acid, e.g., arginine, or an amino sugar, e.g., meglumine, wherein the weight ratio of said decarboxylase inhibitor to said salt of LDA compound is about 1:1 to about 1:100, about 1:2 to about 1:60, about 1:4 to about 1:40, or about 1:10 to about 1:40; and the molar ratio or said decarboxylase inhibitor or salt thereof to said basic amino acid or said amino sugar is about 1:1 to about 1:4, or about 1:1 to about 1:3.5, or about 1:1 to about 1:2.5, and wherein said composition is stable for at least 24 hours at room temperature.

In particular embodiments, the LDA salt comprised within the pharmaceutical composition B of the present invention is a salt of the compound of formula II or III, or an enantiomer, diastereomer, or racemate thereof, e.g., the hydrochloric salt of said LDA compound or an enantiomer, diastereomer, or racemate thereof.

In certain embodiments, the decarboxylase inhibitor comprised within the pharmaceutical composition B of the present invention, as defined in any one of the embodiments above, is selected from carbidopa, benserazide, or a salt thereof; the basic amino acid optionally comprised within said composition is arginine, histidine, or lysine; and the amino sugar optionally comprised within said the composition is meglumine, D-glucosamine, sialic acid, N-acetylglucosamine, galactosamine, or a combination thereof. In particular such embodiments, said decarboxylase inhibitor is carbidopa, said basic amino acid is arginine, and said amino sugar is meglumine. Particular such compositions comprise about 1% or 5%, to about 20%, 25%, or 30%, by weight of said LDA salt; and more particular such compositions are those wherein the LDA salt is a salt of the compound of formula II or III, e.g., the hydrochloric salt of said LDA compound, or an enantiomer, diastereomer, or racemate thereof.

In certain embodiments, the pharmaceutical composition B of the present invention, as defined in any one of the embodiments above, further comprises a buffer. Examples of suitable buffers are provided above and include, e.g., citrate buffer, citric acid buffer, acetate buffer, sodium acetate buffer, acetic acid buffer, tartrate buffer, tartaric acid buffer, phosphate buffer, succinic acid buffer, Tris buffer, glycine buffer, hydrochloric acid buffer, potassium hydrogen phthalate buffer, sodium buffer, sodium citrate tartrate buffer, sodium hydroxide buffer, sodium dihydrogen phosphate buffer, disodium hydrogen phosphate buffer, or a mixture thereof.

In certain embodiments, the pharmaceutical composition B of the present invention, as defined in any one of the embodiments above, further comprises one or more antioxidants. Examples of suitable antioxidants are provided above and include, e.g., ascorbic acid or a salt thereof, a cysteine such as L-cysteine and N-acetyl cysteine (NAC), a bisulfite or a salt thereof, and glutathione.

In certain embodiments, the pharmaceutical composition B of the present invention, as defined in any one of the embodiments above, further comprises a COMT inhibitor, or a MAO inhibitor. Examples of COMT inhibitors and MAO inhibitors are provided above and include, e.g., entacapone, tolcapone, and opicapone; and moclobemide, rasagiline, selegiline, and safinamide, respectively.

In certain embodiments, the pharmaceutical composition B of the present invention, as defined in any one of the embodiments above, further comprises a surfactant. Examples of suitable surfactants are provided above and include, e.g., Tween-80, Tween-60, Tween-40, Tween-20, Tween-65, Tween-85, Span 20, Span 40, Span 60, Span 80, Span 85, polyoxyl 35 castor oil (Cremophor EL), polyoxyethylene-660-hydroxystearate (macrogol 660), or Poloxamer 188 (Pluronic® F-68).

In yet another aspect, the present invention provides an aqueous pharmaceutical composition, also referred to herein as “pharmaceutical composition C”, having a pH of about 3 to about 6, or about 4 to about 5.5, at 25° C., said composition comprising a salt of a LDA compound of the general formula I as defined above, or an enantiomer, diastereomer, or racemate thereof, and a buffer, wherein said composition is stable for at least 24 hours at room temperature.

In particular embodiments, the LDA salt comprised within the pharmaceutical composition C of the present invention is a salt of the compound of formula II or III, or an enantiomer, diastereomer, or racemate thereof, e.g., the hydrochloric salt of said LDA compound or an enantiomer, diastereomer, or racemate thereof.

In certain embodiments, the pharmaceutical composition C of the present invention, as defined in any one of the embodiments above, further comprises one or more antioxidants. Examples of suitable antioxidants are provided above and include, e.g., ascorbic acid or a salt thereof, a cysteine such as L-cysteine and N-acetyl cysteine (NAC), a bisulfite or a salt thereof, and glutathione.

In certain embodiments, the pharmaceutical composition C of the present invention, as defined in any one of the embodiments above, further comprises a COMT inhibitor, or a MAO inhibitor. Examples of COMT inhibitors and MAO inhibitors are provided above and include, e.g., entacapone, tolcapone, and opicapone; and moclobemide, rasagiline, selegiline, and safinamide, respectively.

In certain embodiments, the pharmaceutical composition C of the present invention, as defined in any one of the embodiments above, further comprises a surfactant. Examples of suitable surfactants are provided above and include, e.g., Tween-80, Tween-60, Tween-40, Tween-20, Tween-65, Tween-85, Span 20, Span 40, Span 60, Span 80, Span 85, polyoxyl 35 castor oil (Cremophor EL), polyoxyethylene-660-hydroxystearate (macrogol 660), or Poloxamer 188 (Pluronic® F-68).

According to the present invention, a pharmaceutical composition as defined in any one of the aspects and embodiments above, may further comprise one or more adamantans (e.g., amantadine), nicotinic receptor agonists (e.g., nicotine, galantamine), dopamine receptor agonists (e.g., apomorphine, rotigotine). Such a composition may also comprise an enhancer and/or a gelation agent and/or a thickening agent. Contemplated enhancers include pyrrolidones such as NMP or PVP, polyols, terpenes (nonaromatic compounds found in essential oils, which may be extracted from flowers, fruits, and other natural products), glycerol, lauroglycol, propylene glycol, diethylene glycol monoethyl ether, and/or propylene glycol monocaprylate. Contemplated enhancers include cellulose polymers such as hydroxypropyl cellulose, and/or carbomer polymers and derivatives, e.g., polysaccharides (agarose) polyacrylic polymers, poloxamers, polyvinyl alcohol PVP and mixtures thereof. Non-limiting examples of terpenes include d-limonene, dipentene (d/l-limonene), α-pinene, γ-terpinene, β-mircene, p-cimene, α-pinene, α-phellandrene, citronellolio, geraniale (citrale), nerol, beta-carotene, menthol, geraniol, farnesol, phytol, their homologs, derivatives, enantiomers, isomers including constitutional isomers, stereoisomerisms, regioisomers, and geometric isomers, and any combinations thereof.

In certain embodiments, the pharmaceutical compositions A, B and C disclosed herein, as defined in any one of the embodiments above, may further comprise at least one organic compound, such as ethanolamines, e.g., monoethanolamine, diethanolamine, triethanolamine, phenyl ethanolamine, acetyl ethanolamine, or benzoyl ethanolamine.

The pharmaceutical compositions of the invention are aqueous and may be formulated as a liquid, gel, cream, solid, film, emulsion, suspension, solution, lyophylisate or aerosol, but it is preferably formulated as a liquid. Such compositions may be formulated for any suitable route of administration, e.g., for subcutaneous, transdermal, intradermal, transmucosal, intravenous, intraarterial, intramuscular, intraperitoneal, intratracheal, intrathecal, intraduodenal, intrapleural, intranasal, sublingual, buccal, intestinal, intraduodenally, rectal, intraocular, or oral administration. The compositions may also be formulated for inhalation, or for direct absorption through mucous membrane tissues.

According to the present invention, the pharmaceutical compositions can be administered over a defined time period, e.g., days, weeks, months, or years.

The pharmaceutical compositions of the invention may further comprise a pharmaceutically acceptable carrier. The term “pharmaceutically acceptable carrier” or “pharmaceutically acceptable excipient” as used herein interchangeably refers to any and all solvents, dispersion media, preservatives, coatings, isotonic and absorption delaying agents, and the like, that are compatible with pharmaceutical administration. The use of such media and ingredients for pharmaceutically active substances is well-known in the art. The term “acceptable” with respect to a carrier or an excipient comprised within a pharmaceutical composition refers to any carrier, ingredient or molecular entity that do not produce an adverse, allergic or other untoward reaction when administered to a mammal or human as appropriate. For human administration, compositions should meet sterility, pyrogenicity, and general safety and purity standards as required by, e.g., the U.S. Food and Drug Administration (FDA) or the European Medicines Agency (EMA). It should be understood that the compositions of the invention can also contain active agents in addition to those specifically defined above, so as to provide supplemental, additional, or enhanced therapeutic functions.

The term “physiologically acceptable pH” as used herein means a pH that facilitates administration of the composition to a patient without significant adverse effects, e.g., a pH of about 3 to about 9.5, (for example, about 3.5±0.5 to about 9.0±0.5).

In certain embodiments, the pharmaceutical composition of the present invention is administered substantially continuously, e.g., subcutaneously or transdermally. The term “substantially continuous”, as used herein, means that a single dose of the composition is being administered to said patient or individual over a particular predetermined period of time, e.g., for a period of at least 10, 20 or 30 minutes, 1 hour, 2 hours, 4, hours, 6 hours, 8 hours, 12 hours, 15 hours, 18 hours, 21 hours, 24 hours, 12-16 hours, 16-18 hours, 18-20 hours, or 20-24 hours rather than as a bolus, e.g., as a bolus injection. Substantially continuous administration of these pharmaceutical compositions can be achieved using, e.g., a transdermal patch or a pump device that continuously administers the composition to the patient over time.

In certain embodiments, aqueous pharmaceutical compositions according to the present invention, particularly when comprising a decarboxylase inhibitor or a salt thereof, may be administered at a rate of 0.01 ml/hour/site to 0.4 ml/hour/site, e.g., 0.08 ml/hour/site to 0.24 ml/hour/site. Such rates may be constant throughout the day and night or varied according to patient's need, e.g., may reflect a patient resting or sleeping schedule and waking or higher activity level schedule. Such pharmaceutical compositions may thus be administered, e.g., at a rate of 0.32 ml/hour/site in the morning (e.g., for 2-4 hours before waking), 0.24 ml/hour/site during the daytime or activity time (e.g., for 10 to 12 hours), and/or 0.08 ml/hour/site at rest or at night. In other embodiments, such compositions are administered, e.g., intraduodenally, at a rate of 1.0 ml/hour during the daytime or activity time (e.g., for 2-3 hours before waking and for 10 to 12 hours thereafter), and 0 to 0.5 ml/hour at rest or at night. In further embodiments, such compositions may be administered at a rate of 1.25 ml/hour during the daytime or activity time (e.g., for 2-3 hours before or after waking and for 10 to 14 hours thereafter), and 0 to 0.05 ml/hour (e.g., 0.05±0.005 ml/hour) at rest or night. In still further embodiments, such compositions may be administered at a rate of 0.1 to 1000 μl/hour/site; or at a volume of 2 to 10 ml/24 hour/site, preferably 4 to 6 ml/24 hour/site; or at a dose of 80 to 800 mg levodopa/day and 20 to 200 mg carbidopa/day; or at a rate of 240 to 360 mg levodopa and 60 to 90 mg carbidopa/day/site.

In certain embodiments, a pharmaceutical composition according to the invention may be substantially continuously administered, e.g., using a pump for subcutaneous infusion at an average rate of 10-1000 μl/hour (e.g., 10-250 μl/hour), 300±100 μl/hour, or 200±40 μl/hour continuously for 24 hours; 440±200 μl/hour or 200±50 μl/hour continuously for 16 hours (during waking hours) and 0 to 80 μl/hour or 0 to 200 μl/hour for 8 hours (at night); or using a transdermal patch. Substantially continuously administering the composition to a patient can be doubled or tripled by using more than one pump, patch, or infusion site. In certain embodiments, substantially continuously administering using, e.g., a liquid composition, can be at an average rate of 0.2-2 μl/hour, or 1±0.5 μl/hour continuously for 24 hours; 1±0.5 μl/hour continuously for 16 hours (during waking hours) and 0 to 0.5 μl/hour for 8 hours (at night), via a pump, transdermal patch, or a combination of delivery devices that are suitable for, e.g., subcutaneous, intravenous, intrathecal, and/or intraduodenal administration.

Oral compositions according to the present invention may be prepared according to any method known in the art for the manufacture of pharmaceutical compositions and may be formulated as tablets, pills, dragees, capsules, liquids, gels, syrups, slurries, suspensions, and the like. Such compositions may further comprise one or more ingredients selected from sweetening agents, flavoring agents, coloring agents and preserving agents in order to provide pharmaceutically elegant and palatable preparations. Tablets contain the active ingredients in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate; granulating and disintegrating agents, e.g., corn starch or alginic acid; binding agents, e.g., starch, gelatin or acacia; and lubricating agents, e.g., magnesium stearate, stearic acid, or talc. The tablets may be either uncoated or coated utilizing known techniques to delay disintegration and absorption in the gastrointestinal tract and thereby provide a sustained action over a longer period. For example, a time delay material such as glyceryl monostearate or glyceryl distearate may be employed. They may also be coated using the techniques described in the U.S. Pat. Nos. 4,256,108, 4,166,452 and 4,265,874 to form osmotic therapeutic tablets for control release. The oral compositions may also be in the form of oil-in-water emulsion.

A disclosed composition in the form of a capsule for oral administration may be prepared by filling the suitable gelatin capsule with dry LDA compound of the general formula I, e.g., a compound of formula II or III, and a filler such as methylcellulose or sodium carboxymethyl cellulose, and optionally coating the capsule with enteric coating. Dragee cores are provided with suitable coatings. For this purpose, concentrated sugar solutions may be used which may optionally contain gum arabic, talc, PVP, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions for oral administration include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and optionally stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the compositions are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compositions of the invention may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active ingredients in water-soluble form. Additionally, suspensions of the active ingredients may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the compounds to allow for the preparation of highly concentrated solutions. Alternatively, the active ingredient(s) may be in powder form for constitution with a suitable vehicle, e.g., sterile, pyrogen-free water, before use.

Pharmaceutical compositions for rectal administration may be prepared as suppositories or retention enemas, using for example conventional suppository bases such as cocoa butter or other glycerides.

Pharmaceutical compositions according to the present invention may also be formulated for local administration, such as a depot preparation. Such long acting formulations may be administered by implantation, e.g., subcutaneously or intramuscularly, or by intramuscular injection. Thus, the composition may be formulated, e.g., with suitable polymeric or hydrophobic materials, e.g., as an emulsion in an acceptable oil, or ion exchange resins, or as sparingly soluble derivatives such as sparingly soluble salts.

Formulations for topical administration may include, without limiting, lotions, suspensions, ointments gels, creams, drops, liquids, sprays emulsions and powders. For example, a disclosed composition in the form of gel for topical administration may be prepared by adding sodium metabisulfite, enhancers, e.g., lauroglycol, Capryol 90, and a gelation agent such as hydroxypropyl cellulose, e.g., Klucel HFC or MF grades, to an aqueous solution of a LDA compound of the general formula I, e.g., a compound of formula II or III. A disclosed composition in the form of gel for topical administration may also be prepared by adding sodium metabisulfite, and/or enhancers, and/or gelation agent (e.g. hydroxypropyl cellulose, poly(acrylic)acid, polymethyacrylate (e.g., Carbopol 934P pH 5-6 with or without about 1-5% Eudragit RL-100) to an aqueous solution of a LDA compound as defined hereinabove. A disclosed composition in the form of gel may be prepared by combining said LDA compound, tolcapone, arginine in water, and propylene glycol containing enhancers gelled with hydroxypropyl cellulose, e.g., Klucel HFX.

Contemplated herein, in part, is a dermal patch suitable for transdermal or subcutaneous administration of an active agent that comprises a composition as disclosed herein.

In some embodiments, a pharmaceutical composition as disclosed herein is designed for a slow release of the LDA compound, and therefore includes particles including said compound and a slow release carrier (typically, a polymeric carrier). Slow release biodegradable carriers are well known in the art. These are materials that may form particles that may capture therein an active compound(s) and slowly degrade/dissolve under a suitable environment (e.g., aqueous, acidic, basic, etc.) and thereby degrade/dissolve in body fluids and release the active compound(s) therein. The particles can be, e.g., nanoparticles, i.e., in the range of, e.g., about 1 to about 500 nm, about 50 to about 200 nm, or about 100 nm, in diameter.

Also contemplated herein is a stable lyophilized powder comprising a LDA compound of the general formula I, e.g., the compound of formula II or III, or an enantiomer, diastereomer, racemate, or salt thereof. Such a lyophilized powder can be reconstituted into a liquid formulation by addition of water with or without antioxidants, surfactants etc.

The pharmaceutical compositions of the present invention are useful for treatment of diseases or disorders characterized by neurodegeneration and/or reduced levels of brain dopamine. Such diseases and disorders include neurological or movement diseases, e.g., restless leg syndrome, Parkinson's disease, secondary parkinsonism, Huntington's disease, Parkinson's like syndrome, PSP, MSA, ALS, Shy-Drager syndrome, dystonia, Alzheimer's disease, LBD, akinesia, bradykinesia, and hypokinesia; conditions resulting from brain injury including carbon monoxide or manganese intoxication; and conditions associated with a neurological disease or disorder including alcoholism, opiate addiction, and erectile dysfunction. In a particular embodiment, the disease treated with the pharmaceutical compositions of the invention is Parkinson's disease.

In a further aspect, the present invention relates to a method for treatment of a disease or disorder characterized by neurodegeneration and/or reduced levels of brain dopamine, e.g., neurological or movement diseases such as those listed above, said method comprising administering to a patient, e.g., an individual, in need thereof a therapeutically effective amount of a pharmaceutical composition A as defined in any one of the embodiments above, provided that said composition comprises a decarboxylase inhibitor and at least one of a basic amino acid or an amino sugar; or a pharmaceutical composition B as defined above.

In yet a further aspect, the present invention relates to a method for treatment of a disease or disorder characterized by neurodegeneration and/or reduced levels of brain dopamine, e.g., neurological or movement diseases such as those listed above, said method comprising co-administering to a patient, e.g., an individual, in need thereof (i) a first pharmaceutical composition selected from a pharmaceutical composition A as defined in any one of the embodiments above, provided that said composition does not comprise a decarboxylase inhibitor or a salt thereof, or a pharmaceutical composition C as defined in any one of the embodiments above; and (ii) a second pharmaceutical composition comprising a decarboxylase inhibitor and optionally at least one of a basic amino acid or an amino sugar; and/or a COMT inhibitor; and/or a MAO inhibitor.

In certain embodiments, the second pharmaceutical composition administered according to this method comprises carbidopa or a salt thereof as the decarboxylase inhibitor, and optionally further comprises at least one of arginine as the basic amino acid or meglumine as the amino sugar; entacapone, tolcapone or opicapone as the COMT inhibitor; or moclobemide, rasagiline, selegiline or safinamide as the MAO inhibitor.

In a particular such aspect, the invention relates to a method for treatment of a disease or disorder characterized by neurodegeneration and/or reduced levels of brain dopamine by co-administration of two pharmaceutical compositions as defined above, wherein the first one of said compositions is administered parenterally, intravenously, subcutaneously, intraduodenally, rectally, intrathecally, sublingually, intradermally, intranasally, or intramuscularly; and the second one of said compositions is administered parenterally, intravenously, subcutaneously, transdermally, rectally, intrathecally, sublingually, intradermally, intranasally, intramuscularly, or orally.

In certain embodiments, the invention relates to a method for treatment of a disease or disorder characterized by neurodegeneration and/or reduced levels of brain dopamine by co-administration of two pharmaceutical compositions as defined above, wherein the first one of said compositions and the second one of said composition are administered by the same of different administration routes. Particular such embodiments are those wherein the second one of said compositions are administered orally.

In still a further aspect, the present invention provides a kit for carrying out one of the methods of the invention, said kit comprising (i) a first pharmaceutical composition selected from a pharmaceutical composition A as defined in any one of the embodiments above, provided that said composition comprises neither a decarboxylase inhibitor nor a salt thereof, or a pharmaceutical composition C as defined in any one of the embodiments above; (ii) a second pharmaceutical composition comprising a decarboxylase inhibitor or a salt thereof, and optionally at least one of a basic amino acid or an amino sugar, and/or a COMT inhibitor; and/or a MAO inhibitor; and (iii) optionally instructions for co-administration of said pharmaceutical compositions for treatment of a disease or disorder characterized by neurodegeneration and/or reduced levels of brain dopamine.

The invention will now be illustrated by the following non-limiting Examples.

### EXAMPLES

### Example 1

### The Effect of Buffers and pH Levels on the Stability of LDA-HCl

Liquid formulations were prepared by dissolving LDA-HCl, 50 mg/ml in buffer citrate or buffer phosphate at different pH levels (Table 2). The formulations were incubated 4 days at 37° C., their stability was evaluated by HPLC analysis at the end of the fourth day, and the recovery after 4 days at 37° C. was calculated compared to T=0.

The results in Table 2 show that LDA-HCl is stable for at least 4 days at 37° C. in citrate buffer, but not stable in buffer phosphate at pH>5.7.

TABLE 2

| Buffer | pH | % Recovery after 4 days at 37° C. |
|---|---|---|
| Citrate | 4.1 | 100.2 |
| | 5.1 | 101.9 |
| Phosphate | 5.7 | N/A (Precipitated) |
| | 6.5 | N/A (Precipitated) |
| | 7.4 | N/A (Precipitated) |

Liquid formulations were prepared by dissolving LDA-HCl, 50 mg/ml in water or different buffers (40 mM) at various pH levels. The physical stability of LDA was determined visually after 24 hours and 7 days (Table 3), and the chemical stability of LDA was determined by HPLC analysis (Table 4) after 24 h at room temperature (RT) and expressed as % recovery compared to t₀.

Table 3 clearly shows that the color of the formulations comprising 5% LDA-HCl and water or a buffer depends on the pH of the solution, and that formulations are physically stable for 24 h at room temperature under all conditions tested, but only formulations having a pH<5.5 are stable for at least 7 days. Table 4 shows that formulations comprising 5% LDA-HCl and water or a buffer are chemically stable for at least 24 h at room temperature at pH ranging between 3 to 7.

TABLE 3

| | | Color at | Precipitation at | Precipitation at |
|---|---|---|---|---|
| Buffer | pH | T = 24 h (RT) | T = 24 h (RT) | T = 7 day (RT) |
| Citrate | 3.09 | light yellow | − | − |
| | 3.90 | light yellow | − | − |
| | 4.85 | light yellow | − | − |
| | 5.65 | medium yellow | − | + |
| Acetate | 3.76 | light yellow | − | − |
| | 4.72 | light yellow | − | − |
| Tris | 5.39 | light yellow | − | − |
| | 6.14 | medium yellow | − | + |
| | 6.61 | medium yellow | − | + |
| Phosphate | 6.03 | medium yellow | − | + |
| | 6.32 | medium yellow | − | + |
| Histidine | 6.20 | medium yellow | − | + |
| Tartrate | 3.84 | light yellow | − | − |
| Water for | 3.56 | light yellow | − | − |
| injection | 4.14 | light yellow | − | − |
| (WFI) | 4.95 | light yellow | − | − |
| | 5.93 | medium yellow | − | + |
| | 6.44 | medium yellow | − | + |
| | 6.91 | strong yellow | − | + |

TABLE 4

| | | LDA (mg/ml) | LDA (mg/ml) | LDA |
|---|---|---|---|---|
| Buffer | pH | at T = 0 | at T = 24 hr (RT) | % recovery |
| Citrate | 3.09 | 51.84 | 52.09 | 100.48 |
| | 3.9 | 51.34 | 51.7 | 100.70 |
| | 4.85 | 52.05 | 52.75 | 101.34 |
| | 5.65 | 51.24 | 51.03 | 99.59 |
| Acetate | 3.76 | 52.09 | 52.41 | 100.61 |
| | 4.72 | 52.66 | 53.05 | 100.74 |
| Tris | 5.39 | 51.88 | 52.08 | 100.39 |
| | 6.14 | 51.79 | 51.73 | 99.88 |
| | 6.61 | 51.62 | 51.74 | 100.23 |
| Phosphate | 6.03 | 51.32 | 51.36 | 100.08 |
| | 6.32 | 52.05 | 51.82 | 99.56 |
| Histidine | 6.2 | 51.27 | 51.76 | 100.96 |
| Tartrate | 3.84 | 52.46 | 52.26 | 99.62 |
| WFI | 3.56 | 51.91 | 51.34 | 98.90 |
| | 4.14 | 51.3 | 51.57 | 100.53 |
| | 4.95 | 51.07 | 50.23 | 98.36 |
| | 5.93 | 51.32 | 50.5 | 98.40 |
| | 6.44 | 51.24 | 50.33 | 98.22 |
| | 6.91 | 51.05 | 49.54 | 97.04 |

### Example 2

### Long Term Stability of LDA-HCl

Liquid formulation comprising LDA-HCl in citric buffer was prepared by dissolving 200 mg/ml LDA-HCl in 40 mM citric buffer with 0.05% N-acetylcysteine (NAC) as antioxidant. The formulation was incubated for 2 weeks at 37° C. The stability of LDA after 14 days at 37° C. was evaluated by HPLC analysis, and the recovery calculated compared to t₀ (Table 5).

Table 5 indicates that a formulation comprising high concentrations of LDA-HCl and NAC in citric buffer is stable for at least 14 days.

TABLE 5

| T = 0 | T = 2 w, 37° C. | % Recovery |
|---|---|---|
| 18.69 | 19.01 | 101.67 |

### Example 3

### The Effect of Preparation Method and pH on LDA and CD Stability in LDA-HCl/CD Formulations

LDA-HCl/CD formulations were prepared by the following methods, and were then tested for stability.

Method 1. Carbidopa (CD, powder) [Teva Pharmaceuticals Ltd., Israel] was added to LDA-HCl solution. Heating to 65° C. resulted in complete dissolution of CD. Precipitation occurred at room temperature.

Method 2. Liquid CD formulations were prepared by weighting CD [Teva Pharmaceuticals Ltd., Israel] in a suitable container with different L-arginine [Merck] weights to obtain a final concentration of 0.8% CD (wet form, 0.75% dry form) in different molar ratios to arginine (Table 6). CD formulations were prepared as described in the International Publication No. WO 2010/134074. LDA-HCl (powder) was added to CD solutions to attain the final desired concentration of LDA-HCl/CD formulations.

Method 3. CD solutions (40 mg/ml, prepared as described in the International Publication No. WO 2010/134074) were mixed with LDA-HCl solutions to attain the final desired concentration of LDA/CD formulations. The LDA-HCl formulations were prepared by dissolving 200, 160 or 120 mg/ml of LDA-HCl in 40 mM citric buffer.

As shown, Methods 2 and 3 were suitable for the preparation of LDA-HCl/CD formulations while method 1 yielded unstable formulations.

TABLE 6

| | F1 | F2 | F3 |
|---|---|---|---|
| Arg:CD molar ratio | 1:1.3 | 1:1.6 | 1:1.6 |
| % CD | 0.8 | 0.8 | 4.0 |
| % Arg | 0.74 | 0.92 | 4.62 |
| % Ascorbic acid | 0.1 | 0.1 | 0.5 |
| NAC | 0.03 | 0.03 | 0.135 |
| Water | 98.15 | 98.15 | 90.75 |
| pH | 7.64 | 8.30 | 8.60 |

The chemical stability of 7 LDA-HCl/CD formulations was evaluated by HPLC analysis at t₀ and after 5 days at room temperature. The recovery after 5 days at room temperature was calculated compared to t₀ (Table 7).

Table 7 clearly shows that the chemical stability of CD and LDA is pH-dependent.

### Example 4

### The Effect of CD Concentration and CD:Arg Ratio on the Physical and or Chemical Stability of LDA and CD

Various liquid CD and LDA-HCl formulations were prepared (Tables 8-10) as described in Example 3, following method #2.

Table 8 indicates that LDA-HCl/CD formulations are physically stable at CD/Arg molar ratios ranging from 1:1.3 to 1:2.7. As further shown LDA-HCl:CD formulations having a ratio from about 5:1 to about 30:1 are physically stable for at least 5 days at room temperature.

Tables 9-10 indicate that LDA-HCl/CD formulations are stable at CD/Arg molar ratios ranging from 1:1.25 to 1:3.15. As further shown, in pH ranging from 5.4 to 6.2 and in LDA-HCl:CD ratio from about 10:1 to about 40:1 the LDA-HCl/CD formulations are chemically and physically stable for at least 24 h at room temperature.

TABLE 8

| | CD | Arginine | LDA | | | Physical |
|---|---|---|---|---|---|---|
| | concen- | concen- | concen- | | | stability after |
| | tration | tration | tration | CD:Arg | Final | 5 days (RT) |
| | (%) | (%) | (%) | ratio | pH | (precipitation) |
| F1 | 0.75 | 0.90 | 20 | 1:1.6 | 6.4 | − |
| F2 | 1.75 | 2.15 | 20 | 1:1.6 | NA | + |
| F3 | 0.75 | 0.74 | 20 | 1:1.3 | 6.35 | − |
| F4 | 1.75 | 1.76 | 20 | 1:1.3 | NA | + |
| F5 | 1.75 | 1.76 | 12 | 1:1.3 | 6.78 | + |
| F6 | 1.75 | 1.53 | 12 | 1:1.1 | 6.55 | + |
| F7 | 1.75 | 2.35 | 20 | 1:1.7 | 6.75 | − |
| F8 | 1.75 | 2.64 | 20 | 1:1.9 | 6.85 | − |
| F9 | 1.75 | 3.70 | 20 | 1:2.7 | 6.84 | − |

NA—Not applicable.

### Example 5

### Pharmacokinetic of LDA and LD Following IV and Oral Administration of LDA-HCl in Mice

The purpose of this experiment was to determine the plasma levels of LDA and LD following intravenous (IV) and oral (PO) administration of LDA-HCl in CD-1 mice. The dosing plan is presented in Table 11.

TABLE 11

| | | | |
|---|---|---|---|
| Number of mice | 9 | 15 | 15 |
| Dosing route | IV | PO | PO |
| Test item | LDA-HCl | LDA-HCl | LD/CD |
| Dose (mg/kg) | 20 | 20 | 20/5 |
| Volume (ml/kg) | 2 | 5 | 5 |
| Formulation | 10 mg/ml | 4 mg/ml | 4/1 mg/ml |

CD-1 mice which were prepared for oral administration had to undergo fasting overnight prior to the dosing. The orally treated mice received food only 2 hour post-dosing. The method used to dose orally was oesophagotubage, and the IV mice were injected via a jugular vein under anesthesia (Isoflurane/O₂). LDA-HCl solutions were dissolved in citrate buffer and LD/CD solution was prepared according to International Publication No. WO 2010/134074.

Blood samples were collected following the oral and IV dosing at pre-determined time points and plasma levels of LDA and LD were analysed by LC-MS-MS.

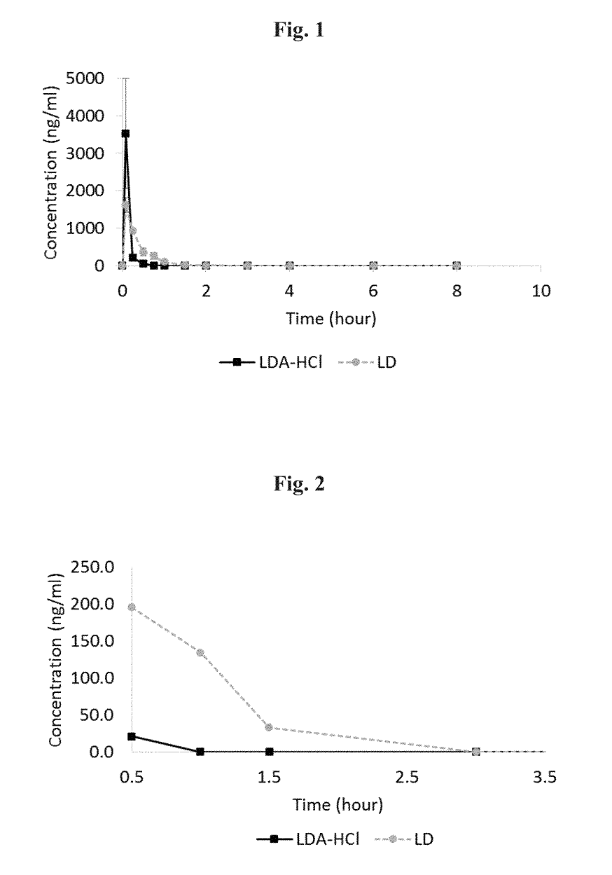

FIG. 1 indicates the plasma concentrations of LDA and LD following IV administration of LDA-HCl (20 mg/kg) in CD-1 mice. The results show that LDA reaches a peak plasma level (Cmax) at the first time point measured after dosing (t=12 minutes) and are below limit of quantification 1 h thereafter. LD plasma levels also reached Cmax by 12 minutes post dosing, and are below limit of quantification 3 h thereafter.

FIG. 2 indicates the plasma concentrations of LDA and LD following oral administration of LDA-HCl (20 mg/kg) in CD-1 mice. The results suggest that LDA is rapidly metabolized to LD following IV and oral administration, and that the half-life of LDA is shorter than that of LD.

The bioavailability of LDA is low when administered orally.

### Example 6

### Pharmacokinetic of LDA and LD Following Continuous Subcutaneous Administration of LDA-HCl in Mice

The purpose of this experiment was to determine the plasma pharmacokinetics of LDA and LD following continuous subcutaneous (SC) administration of LDA-HCl in CD-1 mice. LDA-HCl formulation (170 mg/ml) of was prepared as described above and was continuously administered via Osmotic Alzet pump-#2002 at a rate of 0.5 μl/hr for 3 days. Blood samples were collected 3 days post Alzet pump implantation and plasma levels of LDA and LD were analysed by LC-MS-MS.

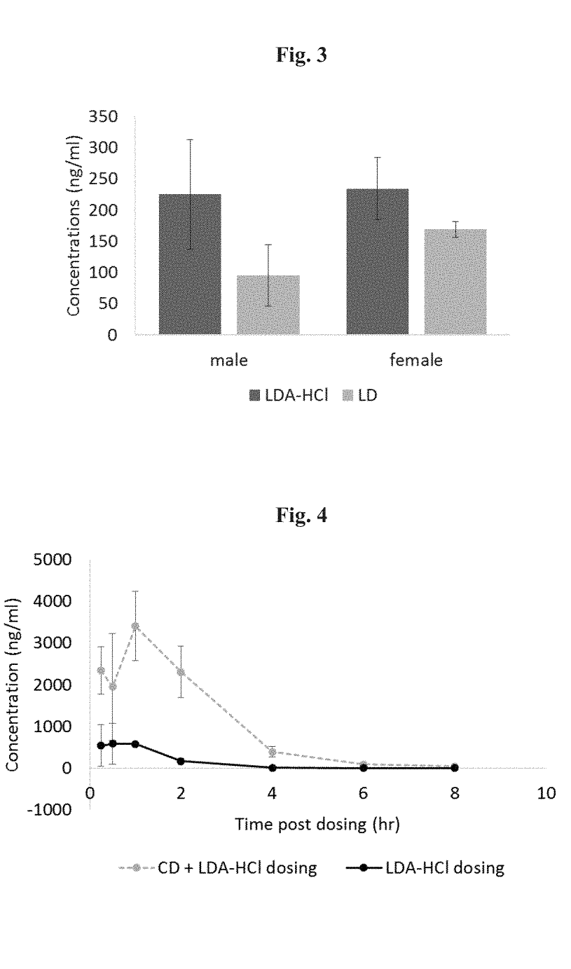

FIG. 3 shows the steady state plasma concentrations of LDA and LD after 3 days of continuous SC administration of LDA-HCl.

### Example 7

### Effect of CD on the Pharmacokinetic of LDA and LD in Rats

The purpose of this experiment was to determine the plasma pharmacokinetics of LDA and LD following oral administration of LD or LDA-HCl with and without CD in Wistar rats.

Wistar rats underwent fasting overnight prior to the dosing and received food only 2 hour post-dosing. The method used to dose orally was via oesophagotubage. Solutions were prepared as described above. The dosing plan is presented in Table 12.

TABLE 12

| | Group 1 | Group 2 | Group 3 |
|---|---|---|---|
| Number of rats | 3 | 3 | 3 |
| API | LDA-HCl | LDA-HCl and CD | LD and CD |
| Dose (mg/kg) | 25 | 10 (CD) | 10 (CD) |
| | | 25 (LDA-HCl) | 25 (LD) |
| Volume (ml/kg) | 10 | 5 (CD) | 5 (CD) |
| | | 10 (LDA-HCl) | 10 (LD) |
| LDA-HCl | 2.5 mg/ml in | 2.5 mg/ml | — |
| formulation | citric buffer | In citric buffer | |
| LD formulation | — | — | 2.5 mg/ml in citric buffer |
| CD formulation | — | 2 mg/ml in water | 2 mg/ml in water |

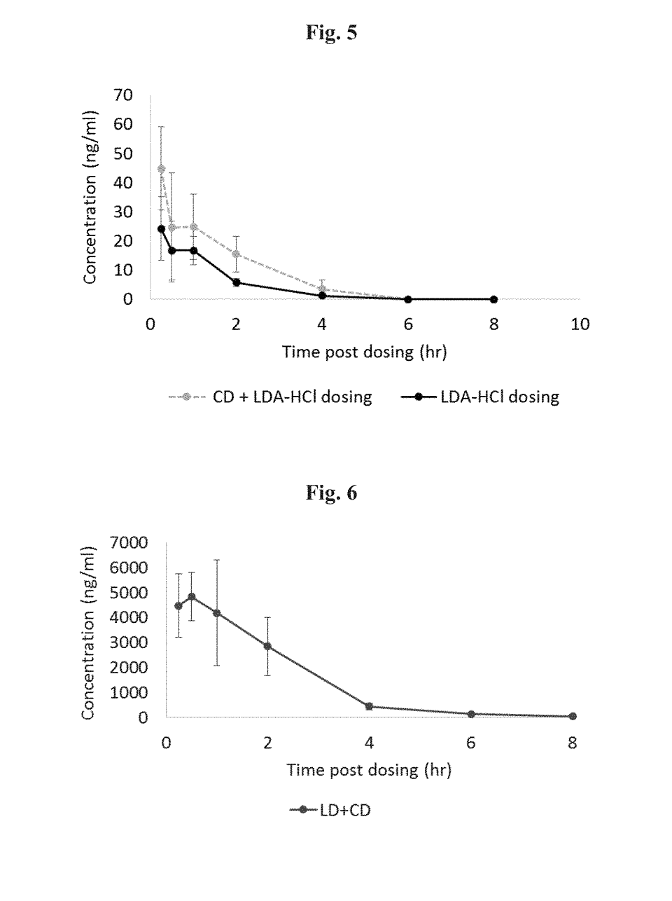

FIG. 4 indicates the plasma concentrations of LD following oral administration of LDA-HCl (25 mg/kg) with or without the oral administration of 10 mg/kg CD and FIG. 5 indicates the plasma concentrations of LDA-HCL following oral administration of LDA-HCl (25 mg/kg) with or without oral administration of 10 mg/kg CD. FIG. 6 shows the plasma concentrations of LD following oral administration of LD/CD. The results show that CD increases the plasma concentration of LD following LDA-HCl administration (FIG. 4) but has no effect on the plasma concentrations of LDA (FIG. 5). The results suggest that LDA is rapidly metabolized to LD and that CD is essential for improving the pharmacokinetic of LD. Therefore, treatment with LDA formulations has to be co-administered with CD.

It is further suggested that LDA requires the co-administration/co-formulation with a decarboxylase inhibitor and/or a COMT inhibitor to improve the pharmacokinetic of LD.

### Example 8

### The Effect of Acids on the Stability of LDA Formulations With/Without CD

The effect of HCl, representing an inorganic acid, on the stability of LDA solution was evaluated. HCl solution containing 0.3% Tween 80 was added to LDA to obtain a solution having a final LDA concentration of 200 mg/ml and a molar ratio of about 1:1 LDA:HCl. The physical stability of the solution and the chemical stability of LDA were evaluated (Table 13). As shown, LDA (200 mg/ml) is stable for at least 9 days in solution containing HCl at a molar ratio of about 1:1.

TABLE 13

| | | | | | | Chemical |
|---|---|---|---|---|---|---|
| LDA:HCl | | Physical stability | | | | stability of LDA |
| molar ratio | pH | T₀ | 24 hrs | 4 days | 9 days | 9 days |
| 1:0 | N/A | + | N/A | N/A | N/A | N/A |
| 1:0.94 | 5.81 | − | − | − | − | 100.1 |

The effect of organic acids on the stability of LDA solution was evaluated. Formulations were prepared by adding small aliquots of organic acid solutions, 100-300 μl, to ˜240 mg LDA until full dissolution while pH was monitored. The end point of acid addition was decided by appearance and/or by pH and/or total volume. LDA concentration was determined by HPLC.

TABLE 14

| | LDA | LDA:acid | | Chemical stability |
|---|---|---|---|---|
| | (mg/ml) | molar ratio | pH | after 3 days at RT |
| Acetic | 199 | 1:1.2 | 5.0 | 98.4 |
| Citric | 236 | 1:08 | 2.8 | 99.3 |
| Succinic | 194 | 1:0.5 | 5.7 | 99.1 |
| Tartaric | 334 | 1:0.6 | 3.8 | 100.5 |
| Glutamic | 128 | 1:1 | 5.8 | N/A |
| Citric | 175 | 1:0.35 | 5.6 | N/A |

As shown in Table 14, LDA can be dissolved in high concentrations and the solution is physically (not shown) and chemically stable for at least 3 days at RT. The molar ratio necessary to dissolve LDA correlates with the number of carboxylic groups for acetic acid (monocarboxylic, 1:1.2), succinic acid (dicarboxylic, 1:0.5), tartaric acid (dicarboxylic, 1:0.6), and citric acid (tricarboxylic acid 1:0.35).

HCl solutions at 3 different concentrations containing 0.3% Tween 80 were added to LDA to obtain solutions having a final LDA concentration of 200 mg/ml and a molar ratio of 1:0.89, 1:0.92 and 1:0.95 LDA:HCl. CD-Arg formulation (CD:Arg molar ratio 1:1.25) was prepared in Tween 80 0.3% and was added gradually under stirring to 4 volumes of each of the 3 LDA-HCl solutions. The physical stability of the solutions was evaluated (Table 15). As shown, the addition of CD to LDA-HCl solutions yielded stable solutions if added to LDA-HCl solutions having a pH>3.8, preferably pH >4.6.

LDA in water with and without 0.3% Tween-80, at concentrations greater than 2.5% (data not shown), is not soluble in the absence of an acid.

TABLE 15

| | | LDA:HCl | CD:Arg | pH | | Precipitations | | |
|---|---|---|---|---|---|---|---|---|
| % | % | molar | molar | LDA-HCl | Final | T = | T = | T = |
| CD | LDA | ratio | ratio | solution | pH | 0 | 24 h | 3 d |
| 00 | 20 | 1:0.90 | 1:1.25 | 5.78 | 5.85 | — | — | — |
| 0.75 | | | | | 6.10 | — | — | — |
| 00 | | 1:0.92 | | 3.70 | 3.77 | — | — | — |
| 0.75 | | | | | 5.69 | Slight | | |
| | | | | | | precipitation | | |
| 00 | | 1:0.93 | | 4.69 | 4.62 | — | — | — |
| 0.75 | | | | | 5.69 | — | — | — |

### EQUIVALENTS

All numbers expressing quantities of ingredients, reaction conditions, and so forth used in the specification are to be understood as being modified in all instances by the term “about”. Accordingly, unless indicated to the contrary, the numerical parameters set forth in this specification are approximations that may vary by up to plus or minus 10% depending upon the desired properties to be obtained by the present disclosure.

### INCORPORATION BY REFERENCE

The entire contents of all patents, published patent applications, websites, and other references cited herein are hereby expressly incorporated herein in their entireties by reference.

1. An aqueous pharmaceutical composition having a pH of about 3 to about 7 at 25° C., said composition comprising a levodopa amide (LDA) compound of the general formula I: 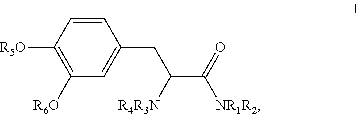
or an enantiomer, a diastereomer, or a racemate thereof,
wherein:
R₁, R₂, R₃ and R₄ each independently is H, (C₁-C₆)alkyl, (C₂-C₆)alkenyl, (C₂-C₆)alkynyl, cycloalkyl, aryl, —O—C(═O)—R′, —C(═O)—OR′, —C(═O)—R′, —C(═S)—R′, —O—C(═O)—NR′R′, —O—C(═S)—NR′R′, or —O—C(═O)—R″, or R₁ and R₂ together with the nitrogen atom to which they are attached form a 5- or 6-membered ring, or R₃ and R₄, together with the nitrogen atom to which they are attached form a 5-membered ring or a 6-membered ring,
R₅ and R₆ each independently is H, (C₁-C₃)alkyl, cycloalkyl, phenyl, or —P(═O)(OR′)₂,
R′ each independently is H, (C₁-C₆)alkyl, (C₂-C₆)alkenyl, cycloalkyl, aryl, or heteroaryl bonded through a ring carbon, and
R″ is a saturated or unsaturated hydrocarbon chain having at least 10 carbon atoms; and
an acid having n acidic groups, wherein n is an integer of 1 or more,
wherein the molar ratio of said LDA compound to said acid is about 1:1/n to about 1:≧1.1, and said composition is stable for at least 24 hours at room temperature.

2. The pharmaceutical composition of claim 1, comprising about 1% to about 30%, or about 5% to about 20%, by weight of said LDA compound.

3. The pharmaceutical composition of claim 1, wherein said LDA compound is 2-amino-3-(3,4-dihydroxyphenyl)propanamide, or an enantiomer, a diastereomer, or a racemate thereof.

4. The pharmaceutical composition of claim 1, wherein said acid is selected from the group consisting of methanesulfonic acid, ethanesulfonic acid, benzenesulfonic acid, p-toluenesulfonic acid, maleic acid, malic acid, fumaric acid, tartaric acid, benzoic acid, acetic acid, citric acid, ascorbic acid, lactic acid, gluconic acid, formic acid, oxalic acid, succinic acid, glutamic acid, aspartic acid, hydrochloric acid, hydrobromic acid, phosphoric acid, sulfuric acid, carbonic acid, and any combination of the aforesaid.

5. The pharmaceutical composition of claim 1, further comprising a decarboxylase inhibitor, and at least one of a basic amino acid or an amino sugar.

6. (canceled)

7. The pharmaceutical composition of claim 5, wherein said decarboxylase inhibitor is carbidopa; said basic amino acid is arginine; and said amino sugar is meglumine.

8. The pharmaceutical composition of claim 5, wherein the weight ratio of said decarboxylase inhibitor to said LDA compound is about 1:1 to about 1:100, about 1:2 to about 1:60, about 1:4 to about 1:40, or about 1:10 to about 1:40; or the molar ratio of said decarboxylase inhibitor to said basic amino acid or said amino sugar is about 1:1 to about 1:4, about 1:1 to about 1:3.5, or about 1:1 to about 1:2.5.

9. The pharmaceutical composition of claim 1, further comprising a buffer.

10. The pharmaceutical composition of claim 9, wherein said buffer is selected from the group consisting of citrate buffer, citric acid buffer, acetate buffer, sodium acetate buffer, acetic acid buffer, tartrate buffer, tartaric acid buffer, phosphate buffer, succinic acid buffer, Tris buffer, glycine buffer, hydrochloric acid buffer, potassium hydrogen phthalate buffer, sodium buffer, sodium citrate tartrate buffer, sodium hydroxide buffer, sodium dihydrogen phosphate buffer, disodium hydrogen phosphate buffer, and a mixture thereof.

11. The pharmaceutical composition of claim 1, further comprising at least one antioxidant.

12. The pharmaceutical composition of claim 11, wherein said antioxidant each independently is selected from the group consisting of ascorbic acid or a salt thereof, a cysteine, a bisulfite or a salt thereof, and glutathione.

13. The pharmaceutical composition of claim 1, further comprising a catechol-O-methyl transferase (COMT) inhibitor, or a monoamine oxidase (MAO) inhibitor.

14. (canceled)

15. The pharmaceutical composition of claim 1, further comprising a surfactant.

16. An aqueous pharmaceutical composition having a pH of about 3 to about 9.5, or about 4 to about 8, or about 5 to about 7, or about 5.5 to about 6.5, at 25° C., said composition comprising a salt of a levodopa amide (LDA) compound of the general formula I: 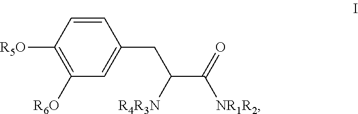
or an enantiomer, a diastereomer, or a racemate thereof,
wherein:
R₁, R₂, R₃ and R₄ each independently is H, (C₁-C₆)alkyl, (C₂-C₆)alkenyl, (C₂-C₆)alkynyl, cycloalkyl, aryl, —O—C(═O)—R′, —C(═O)—OR′, —C(═O)—R′, —C(═S)—R′, —O—C(═O)—NR′R′, —O—C(═S)—NR′R′, or —O—C(═O)—R″, or R₁ and R₂ together with the nitrogen atom to which they are attached form a 5-membered ring or a 6-membered ring, or R₃ and R₄, together with the nitrogen atom to which they are attached form a 5- membered ring or a 6-membered ring,
R₅ and R₆ each independently is H, (C₁-C₃)alkyl, cycloalkyl, phenyl, or P(═O)(OR′)₂,
R′ each independently is H, (C₁-C₆)alkyl, (C₂-C₆)alkenyl, cycloalkyl, aryl, or heteroaryl bonded through a ring carbon, and
R″ is a saturated or unsaturated hydrocarbon chain having at least 10 carbon atoms;
a decarboxylase inhibitor or a salt thereof; and
optionally at least one of a basic amino acid or an amino sugar,
wherein the weight ratio of said decarboxylase inhibitor to said salt of LDA compound is about 1:1 to about 1:100, about 1:2 to about 1:60, about 1:4 to about 1:40, or about 1:10 to about 1:40; and the molar ratio or said decarboxylase inhibitor or salt thereof to said basic amino acid or said amino sugar is about 1:1 to about 1:4, or about 1:1 to about 1:3.5, or about 1:1 to about 1:2.5,
and wherein said composition is stable for at least 24 hours at room temperature.

17. The pharmaceutical composition of claim 16 comprising about 1% to about 30%, or about 5% to about 20%, by weight of said salt of LDA compound.

18. The pharmaceutical composition of claim 16, wherein said LDA compound is 2-amino-3-(3,4-dihydroxyphenyl)propanamide, or the hydrochloric salt thereof.

19-20. (canceled)

21. The pharmaceutical composition of claim 16, wherein said decarboxylase inhibitor is carbidopa; said basic amino acid is arginine; and said amino sugar is meglumine.

22-28. (canceled)

29. An aqueous pharmaceutical composition having a pH of about 3 to about 6, or about 4 to about 5.5, at 25° C., said composition comprising about 5% to about 20% by weight of a salt of 2-amino-3-(3,4-dihydroxyphenyl)propanamide
a buffer,
wherein said composition is stable for at least 24 hours at room temperature.

30-38. (canceled)

39. The pharmaceutical composition of claim 29, formulated for subcutaneous, transdermal, intradermal, transmucosal, intravenous, intraarterial, intramuscular, intraperitoneal, intrathecal, intrapleural, intratracheal, intranasal, sublingual, buccal, intestinal, intraduodenal, rectal, or intraocular administration.

40-48. (canceled)
